(19) United States
(12) Patent Application Publication
(10) Pub. No.: US 2015/0230746 A1
(43) Pub. Date: Aug. 20, 2015

(54) Fabric Sleeve for Niddah Inspection

(71) Applicant: Elazar Winberg, Adi (IL)

(72) Inventor: Elazar Winberg, Adi (IL)

(21) Appl. No.: 14/701,536

(22) Filed: May 1, 2015

Related U.S. Application Data
(63) Continuation-in-part of application No. 13/858,051, filed on Apr. 7, 2013.

(30) Foreign Application Priority Data
Apr. 19, 2012 (IL) 219280

(51) Int. Cl. A61B 5/00

(52) U.S. Cl. CPC A61B 5/4337

(57) ABSTRACT

A fabric sleeve for Niddah inspection into which a finger of a user may be inserted. The sleeve is made up of a tubular piece of fabric and a removal means and a securing means. The tubular piece of fabric is closed at its bottom end and has an upper opening, into which the finger is inserted. The removal means is attached to the bottom end of the tubular piece of fabric. The securing means is attached to a top part of the tubular piece of fabric.

### RELATED APPLICATIONS

This application is a continuation in part of U.S. patent application Ser. No. 13/858,051 filed on 7 Apr. 2013 which claimed priority of an Israeli Patent Application number 219280 filed on 19 Apr. 2012.

### TECHNICAL FIELD The present invention refers to a fabric sleeve for inspecting Niddah according to the Halakhah (Jewish rabbinical law).

### BACKGROUND ART

According to the laws of family purity, which are part of the Jewish Halakhah (religious laws), a woman may engage in intercourse only after seven “clean” days have passed since the end of her monthly period. Thus, a Kosher, Halakhah-observant woman must conduct internal inspections every day to see whether there is any blood in her vagina (hereinafter “the inspection” or “the Niddah inspection”). The Niddah inspection is customarily performed using a piece of fabric that the woman wraps around her finger. When in doubt, the woman must show the piece of fabric to a rabbi so that he can determine whether the staining on the fabric is blood or other secretions. It is very important to keep the fabric in the same condition it was in when it was removed from the vagina so as not to disturb the findings. Since the inspection is performed using a piece of fabric that is usually rectangular in shape and is wrapped around the finger, creases and folds often form in the fabric, making it difficult to determine the nature of the findings. Sometimes such creases and folds even accidentally cause internal injury or scratching in women with sensitive skin. Needless to say, it is also quite inconvenient to use a piece of fabric to perform the inspection. The present invention offers an efficient and suitable solution to these problems.

### THE DRAWINGS

The drawings attached to the present application are not intended to limit the scope of the invention and the possible ways of its application The drawings are intended only to illustrate the invention and constitute only one of many possible ways of its application.

FIG. 1 depicts the sleeve (1) with a closed bottom end (11) and an upper opening (12) at the top part (13).

FIG. 2 depicts the sleeve (1) with a removal means (2) in the form of a protrusion (21).

FIG. 3 depicts the sleeve (1) with a removal means (2) in the form of a piece of thread (22).

FIG. 4 depicts the sleeve (1) with a closable opening in closed position.

FIGS. 5-7 describe the sleeve (1) that includes a securing means (14).

### THE INVENTION

The main objective of the present invention is to provide a fabric sleeve (1) that is designed to enable observant Jewish women to perform the Niddah inspection. The sleeve (1) is closed at one end and open at the other so that the sleeve may be inserted over a finger in order to perform the inspection. Another objective of the present invention is to provide a sleeve (1) as mentioned whereby its shape enables it to be removed from the finger after the inspection without turning it inside out, i.e. so that the findings remains on the outside of the sleeve since the sleeve is not turned inside out.

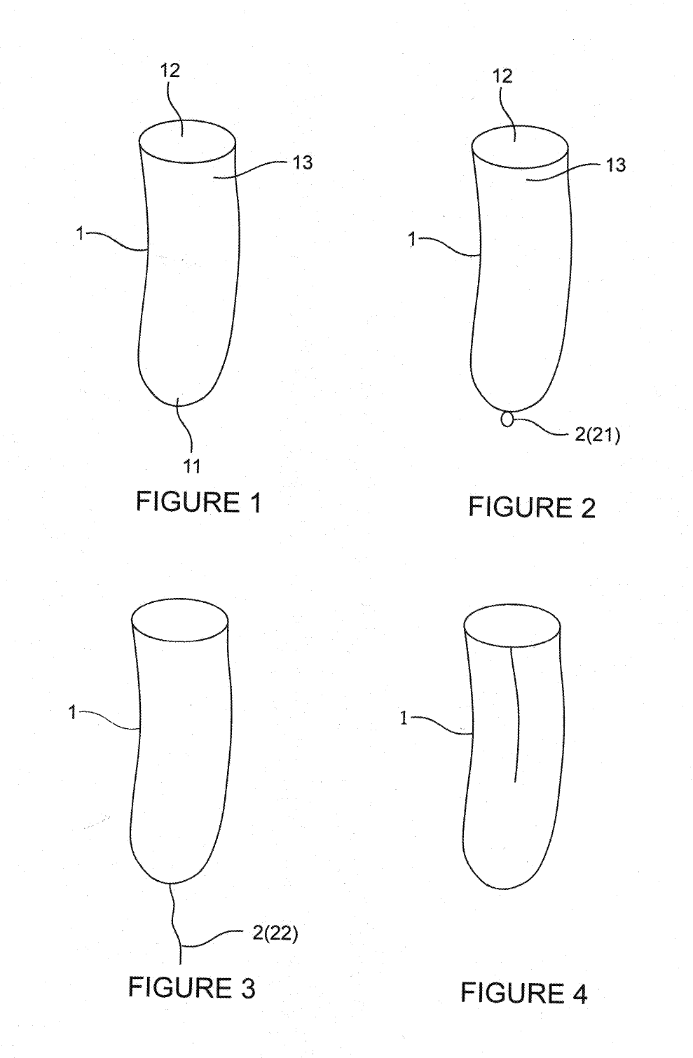

The basic design of the sleeve (1) is depicted in FIG. 1. The sleeve comprises a tubular piece of fabric that is closed at the bottom end (11) and has an upper opening (12) at the upper end into which the user inserts her finger in order to perform the inspection. The sleeve (1) may be shaped like a finger of a glove and it can be made in a variety of sizes to fit a variety of standard finger sizes, as is customary in the manufacture of standard gloves. The size of the sleeve (1) should be such that it stretches over the finger when it is put on, so that no folds or creases form during use.

Using the sleeve (1): The user inserts her finger into the sleeve (1), performs the internal inspection, extracts the finger along with the sleeve (1), and removes the sleeve (1) from the finger without turning it inside out, so that any findings on the sleeve (1) will remain on the outside and will not be disturbed by turning the sleeve (1) inside out. The importance of being able to remove the sleeve (1) from the finger without turning it inside out is hereby emphasized.

The sleeve (1) can and should be designed so that its top part (13) is relatively wide, so as to facilitate its removal from the finger without turning it inside out. Thus, in order to remove the sleeve (1), the user can simply insert a finger between the sleeve (1) and the finger it is on, and push the sleeve (1) off the finger without turning it inside out.

The sleeve (1) is equipped with a removal means (2) to facilitate the removal of the sleeve (1) from the finger without turning it inside out. The removal means (2) may be a protrusion (21) located on the bottom end (11) of the sleeve (1), as depicted for instance in FIG. 2. Alternatively, the removal means (2) may be a piece of string (22) that is attached to the bottom end (11) of the sleeve (1) so as to facilitate its removal from the finger, as depicted for instance in FIG. 3.

Another possible solution to the aforementioned problem is to design the sleeve (1) so that the upper opening (13) and may be closed using a weak adhesive and opened upon completion of the inspection. FIG. 4 depicts the sleeve (1) with the said opening in closed position.

The sleeve (1), subject of the present invention, enables the user to perform the Niddah inspection in an easy, convenient and efficient manner. The part of the sleeve (1) that comes in contact with the inspected area has no creases or folds, thus preventing internal injury and scratches in women with sensitive skin. The sleeve (1) may be very easily removed from the finger without turning it inside out and thus without disturbing the findings.

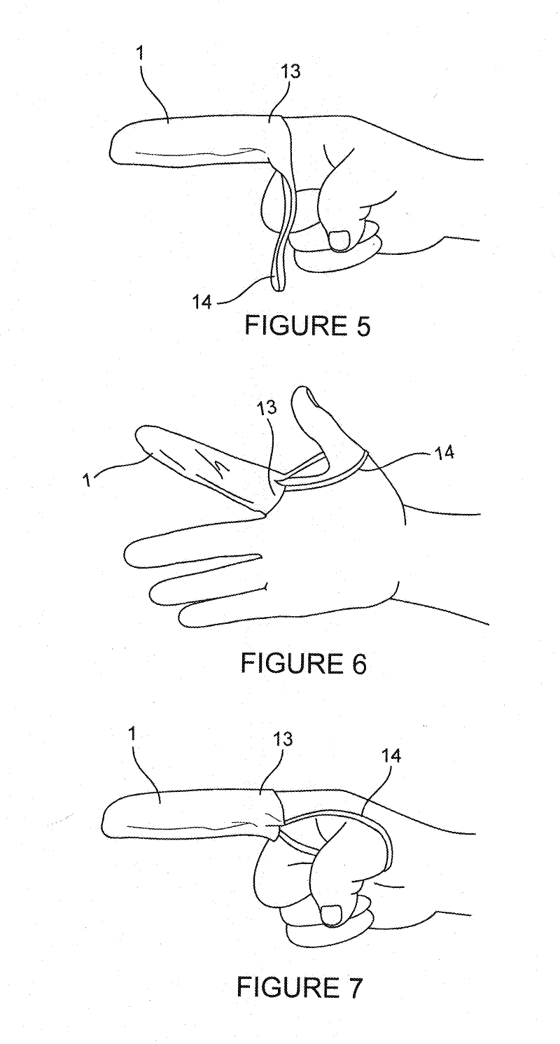

A second version of the invention relates to the sleeve (1) as described above which further includes a securing means (14). The securing means (14) is designed to secure the sleeve (1) during performance of the inspection and to prevent the risk that the sleeve (1) will be unintentionally removed from the finger while pulling it out from the vagina. The securing means (14) is attached to the top part (13) of the sleeve (1). The securing means (14), for example, can be a securing thread which one end of it is attached to the top part (13) of the sleeve (1) or a securing loop which is attached to the top part (13) as described for example in FIGS. 5-7. During the performance of the inspection the user can hold the securing means (14). Alternatively, the securing means (14) can be attached to the hand or a finger of the user. In case when the securing means (14) is in a type of a securing loop, then the user can put the sleeve (1) on her finger, as described in FIG. 5, then the user can put the securing loop on her thumb as described in FIG. 6, and then the finger is ready to perform the Niddah inspection, as described in FIG. 7.

When the user pulls out the finger during the inspection she needs to hold the securing means (14) in purpose that the sleeve (1) will remain smooth and without creases and folds. As explained above, it is very important to keep the fabric sleeve in the same condition it was in when it was removed from the vagina so as not to disturb the findings.

What is claimed is:

1. A fabric sleeve for Niddah inspection into which a finger of a user may be inserted, comprises a tubular piece of fabric and a removal means; wherein said tubular piece of fabric is closed at its bottom end; wherein said tubular piece of fabric has an upper opening, into which said finger is inserted; wherein said removal means is attached to said bottom end of said tubular piece of fabric; whereby enable said user to remove said sleeve from said finger in the end of said Niddah inspection without turning said sleeve inside out.

2. A fabric sleeve for Niddah inspection into which a finger of a user may be inserted, comprises a tubular piece of fabric and a securing means; wherein said tubular piece of fabric is closed at its bottom end; wherein said tubular piece of fabric has an upper opening, into which said finger is inserted; wherein said securing means is attached to a top part of said tubular piece of fabric; wherein said securing means enables said user to pull out said finger in the end of said Niddah inspection without turning said sleeve inside out.

3. The fabric sleeve according to claim I wherein said securing means is a securing loop.
